(19) United States
(12) Patent Application Publication
(10) Pub. No.: US 2013/0158608 A1
(43) Pub. Date: Jun. 20, 2013

(54) PLATE AND CANNULATED TRANSFIXATION SCREW SYSTEM FOR HUMAN IMPLANT

(71) Applicants: RANDY VIOLA, Vail, CO (US); Jennifer Bender, Dallas, TX (US); Brandon Beckendorf, Carrollton, TX (US); Viorel Mocanu, Lewisville, TX (US)

(72) Inventors: RANDY VIOLA, Vail, CO (US); Jennifer Bender, Dallas, TX (US); Brandon Beckendorf, Carrollton, TX (US); Viorel Mocanu, Lewisville, TX (US)

(73) Assignee: OsteoMed L.P., Addison, TX (US)

(21) Appl. No.: 13/331,881

(22) Filed: Dec. 20, 2011

(51) Int. Cl. A61B 17/80; A61B 17/86

(52) U.S. Cl. 606/289; 606/315

(57) ABSTRACT

A system for securing first and second bones together across a joint includes a fixation plate comprising a body having a longitudinal axis and first and second ends. A locking screw hole is disposed proximate to the first end. One or more attachment screw holes are disposed in the body. The system includes a screw comprising an elongate hollow body having openings at first and second ends and having a tip at the first end and a head at the second end.

### TECHNICAL FIELD

The invention generally relates to orthopedic implants, and more particularly to a plate and cannulated transfixation screw system for fixation of bones across a joint in a human hand.

### BACKGROUND

Fractured or broken bones are often repaired by holding bone fragments together to achieve fusion. Also, rheumatoid arthritis, a chronic autoimmune disease, often causes destruction of joints, which requires reconstruction by fusion of bones across a joint. In a human finger, three bones are separated by two joints, called interphalangeal (IP) joints. The joint near the end of the finger is called a distal interphalangeal (DIP) joint and the joint near the hand is called a proximal interphalangeal (PIP) joint. If, for example, a bone across a DIP joint or a PIP joint is fractured, the bone fragments may be held together by an implant device such as, for example, a Kirschner wire (K-wire), a tension band wire, or a compression screw. A K-wire is a thin, rigid wire that is used to stabilize bone fragments. A K-wire can be drilled through a bone and through a joint to hold the fragments in place. A disadvantage of a K-wire is that it generally lacks structural rigidity. Consequently, bones fused across a joint with a K-wire often do not achieve a necessary degree of fusion. More importantly, a K-wire does not generally allow bones across a joint to be fused together in a configuration that provides versatility and improved usage.

A tension band wire uses tensile force to compress and hold bone fragments together. Since a tension band wire lacks structural rigidity, it does not allow bones across a joint to be fused in a configuration that provides versatility and improved usage.

Although compression screws provide the necessary degree of fusion, they typically limit the angle at which bones across a joint may be fused. For example, bones across a PIP joint of a finger preferably should be fused at an angle between 20 and 50 degrees for improved quality of life. In order to fuse bones within the desired range, a compression screw must be placed through the shaft of a bone, which may cause the screw to break out through the dorsal surface. Fixation plate and screw systems have been used to repair fractured bones across a joint. A fixation plate is typically placed upon bones across a joint, and the plate is thereafter secured to the bones by a plurality of attachment screws. While existing fixation plate and screw systems generally achieve the necessary degree of fusion, these systems typically do not allow bones across a joint to be fused in a configuration that provides improved quality of life.

### SUMMARY

Embodiments of the invention provide systems and methods which allow bones across a joint to be fused at a functional angle and provides for an improved quality of usage while also providing sufficient degree of fusion. Embodiments may include a transfixation screw having an elongate hollow body, which is configured to be inserted through a locking screw hole of a plate and extend through a first bone across a joint and into a second bone. Also, embodiments may include a fixation plate having a locking screw hole which will allow for an angle that is ideal for a specific joint.

In one embodiment a screw includes an elongate hollow body having openings at first and second ends and having a tip at the first end and a head at the second end. The body is configured to extend through a first bone across a joint and into a second bone. A first thread originates from the tip and extends proximate from the tip. The first thread has a first pitch. A second thread is disposed proximate to the head and extends distally from the head. The second thread has a second pitch. The second thread is preferably separated from the first thread by an unthreaded section of the body. A locking thread is disposed on the head proximal to the second thread. The locking thread has a third pitch.

In another embodiment a fixation plate includes a body having a longitudinal axis and first and second ends. The plate includes a locking screw aperture disposed proximate to the first end. The aperture includes an inner surface having an upper countersink and a threaded portion below the upper countersink. The threaded portion has double helical threads configured to engage the screw. The aperture has a central axis oriented at an angle between 20 to 50 degrees relative to the longitudinal axis of the body. One or more additional apertures are disposed within the body to accommodate additional attachment screws.

In yet another embodiment of the invention, a system comprises the fixation plate and the screw which transfixes across a joint to be fused. The fixation plate is configured to be placed upon a first bone proximate a joint. The fixation plate may be contoured proximate the first end to match the contour of the first bone. The screw has a first recessed region proximate to the tip to form a first cutting flute and a second recessed region proximate to the head to form a second cutting flute. The first thread has a different pitch than the second thread. The locking thread and the second thread may be separated or may be connected.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1A illustrates a top view of a fixation plate of the present invention.

FIG. 1B illustrates a side view of the fixation plate of FIG. 1A.

FIG. 2 illustrates an enlarged view of a locking screw thread of the present invention.

FIG. 3A is a side view of a cannulated transfixation screw.

FIG. 3B is a side sectional view of a cannulated transfixation screw.

FIG. 4 illustrates a fixation plate and a screw used to fixate bones across a joint.

FIG. 5 illustrates a fixation plate in accordance with another embodiment of the present invention.

### DETAILED DESCRIPTION OF THE INVENTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details. In some instances, well-known structures and components are shown in block diagram form in order to avoid obscuring such concepts.

The word “exemplary” is used herein to mean “serving as an example, instance, or illustration.” Any embodiment or aspect described herein as “exemplary” is not necessarily to be construed as preferred or advantageous over other embodiments or aspects. In addition, references to “an,” “one,” “other,” or “various” embodiments or aspects should not be construed as limiting since various aspects of the disclosed embodiments may be used interchangeably within other embodiments.

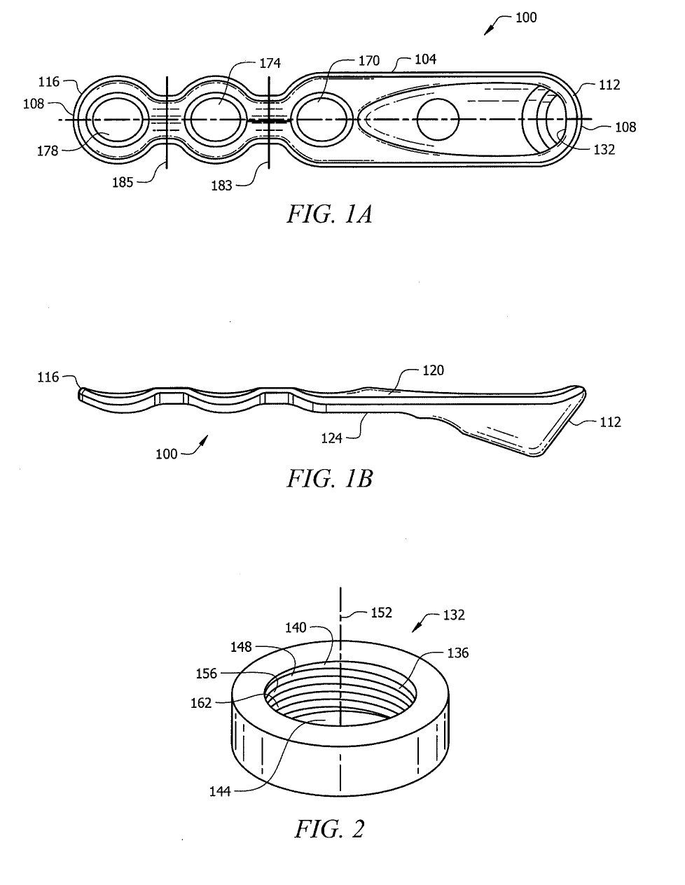

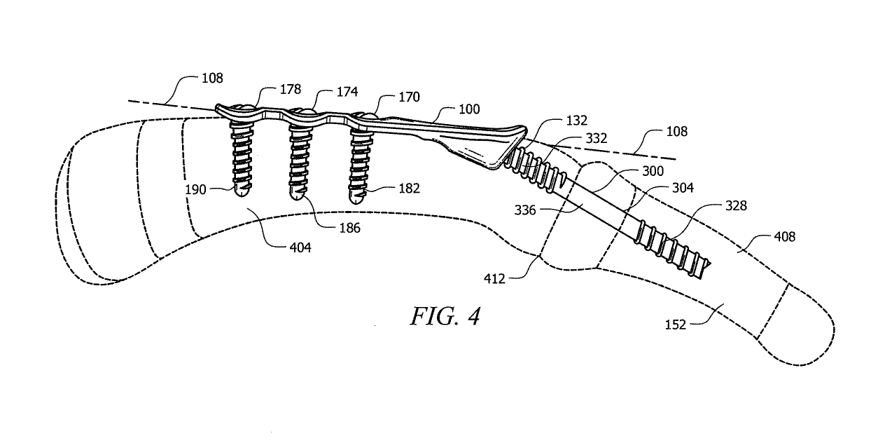

Referring to FIGS. 1A and 1B, fixation plate 100 comprises a body 104 having a longitudinal axis 108 and first and second ends, 112 and 116, respectively. FIG. 1B illustrates a side view of fixation plate 100 having top and bottom sides 120 and 124, respectively. Top side 120 of fixation plate 100 is intended to face away from a bone while bottom side 124 is intended to be placed upon a bone. Portion of bottom side 124 may be contoured to match the contour of a bone on which it is placed. For example, as shown in FIG. 1B, fixation plate 100 may be curved on one side proximate to first end 112 to enable plate 100 to partially section a bone as shown in FIG. 4. In some embodiments, fixation plate 100 is configured to be placed upon a bone and positioned on one side of a joint.

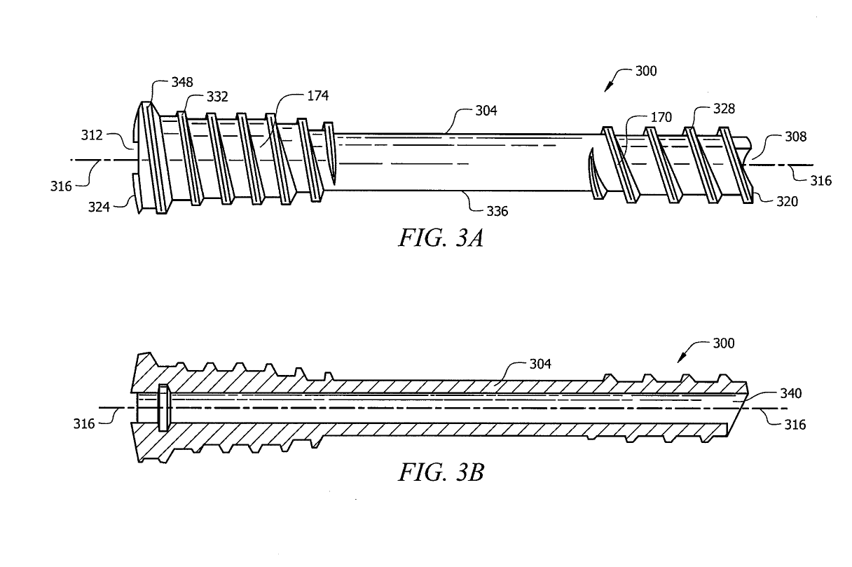

Referring now to FIGS. 1A, 1B, and 2, Fixation plate 100 includes an aperture 132 disposed proximate to first end 112. Aperture 132 may be referred to herein as a locking screw hole 132. However, in other embodiments of the invention screw hole 132 may not be locking. FIG. 2 illustrates an enlarged view of locking fixation screw hole 132 that may be included within fixation plate 100. Locking screw hole 132 may be defined by an inner surface 136 having upper counter sink 140, lower counter sink 144 and threaded portion 148 between upper counter sink 140 and lower counter sink 144. A central axis 152 defines the orientation of locking fixation screw hole 132. Locking fixation screw hole 132 is configured to receive a locking fixation screw (as shown in FIGS. 3A and 3B for example) whose trajectory is defined by central axis 152. In some embodiments of the invention, central axis 152 has an angle between 20 and 50 degrees relative to longitudinal axis 108. Consequently, a screw will be finally positioned at an angle between 20 and 50 degrees relative to longitudinal axis 108, which allows the bones across a joint to be fused in a configuration that provides improved versatility. Referring still to FIG. 2, threaded portion 148 may, for example, include a pair of threads 156 and 162 arranged in a double helix configuration so that threads 156 and 162 may deform when lockably engaging a locking screw, thus enabling the locking screw to lock on to fixation plate 100. Also, pair of threads 156 and 162 enable a locking screw to engage transfixation screw hole 132 at an angle other than parallel to central axis 152. In some embodiments, pair of threads 156 and 162 enable a locking transfixation screw to engage screw hole at an angle between 0 and 18 degrees relative to central axis 152. It should be apparent to those skilled in the art that threads 156 and 162 allow the trajectory of the locking transfixation screw relative to central axis 152 to be adjusted between 0 and 18 degrees giving the surgeon flexibility when installing the transfixation screw. Furthermore, although transfixation screw hole 132 is shown to have a locking mechanism comprising pair of threads 156 and 162, it will be apparent to those skilled in the art that transfixation screw hole 132 may feature any other suitable locking mechanism.

Fixation plate 100 includes one or more attachment screw holes 170, 174 and 178. Attachment screw holes 170, 174 and 178 are configured to receive attachment screws (see screws 182, 186, and 190 in FIG. 4) which may be locking or non-locking. The attachment screws firmly anchor fixation plate 100 to the bone upon which it is placed. In some embodiments, attachment screw holes are threaded to lockably engage attachment screws. Depending on the length of a particular bone upon which fixation plate 100 is placed upon, the length of fixation plate 100 may shortened by sawing or snipping off a portion, for example, along lines 183 or 185, which may also reduce the number of attachment screw holes. In some embodiments, the width of fixation plate 100 may be reduced within the region of lines 183 and 185 to allow fixation plate 100 to be easily sawed or snipped off along lines 174 or 178. Fixation plate 100 is made preferably from stainless steel, titanium or any biocompatible material. The dimension and shape of plate 100 may vary depending on the purpose. As discussed before, in some embodiments, fixation plate 100 is configured to be placed upon a bone on one side of a joint. In other words, fixation plate 100 is placed so that it does not span the joint. Consequently, attachment screws 17, 174 and 178 are inserted into a bone and resides only on one side of the joint. In some embodiments, it may be preferable for the plate to be positioned so that it enters into, or spans, the joint.

Referring to FIG. 3A, a transfixation screw 300 in accordance with embodiments of the invention is shown having an elongated hollow body 304 with openings on first and second ends, 308 and 312, respectively. Screw 300 has a central or longitudinal axis 316 running through hollow body 304. Tip 320 is located at first end 308 and head 324 is located at second end 312.

Referring still to FIG. 3A, screw 300 has first thread 328 originating from tip 320 and extending proximate from tip 320, and second thread 332 disposed proximate to head 324 and extending distally from head 324. Preferably, first thread 328 has a different pitch than second thread 332. For example, first thread 328 may have a larger pitch than that of second thread 332. First and second threads 328 and 332 may be separated by a lag portion (also referred to as an unthreaded portion 336). It will be understood by those skilled in the art that by having different pitches of first and second threads, 328 and 332, and unthreaded portion 336, enables screw 300 to function as a compression-type screw which is particularly suitable for bone fixation. That is, by having at least two different pitches for the two threads the screw wants to advance a different longitudinal distance with each rotation of the screw. Such does not occur, rather the bone portions that the two threaded sections are engaging are put into compression which is the desired result to promote healing at a fracture.

Referring now to FIG. 3B, elongated hollow body 304 has a channel 340 along central axis 316. Channel 340 allows transfixation screw 300 to be correctly positioned relative to a bone by threading a guide wire (e.g., K-wire) through channel 340, a practice well known to those skilled in the art. For example, a surgeon may first insert a K-wire into a desired location and then slide transfixation screw 300 over the K-wire to properly position transfixation screw 300.

Referring back to FIG. 3A, head 324 may include a locking thread 348. Locking thread 348 may have a pitch that is different from the pitch of second thread 328. Locking thread 348 may be continuous with second thread 328 or may be separated from second thread 328. Locking thread 348 is configured to engage pair of threads 156 and 162 disposed on transfixation screw hole 132 as discussed with respect to FIG. 2. Thus, locking thread 348 would be configured to deform upon engagement with threads 156 and 162 thereby lockably engaging fixation plate 100. According to embodiments of the invention, locking thread 348 and threads 156/162 are made of different materials so that one is softer than the other thereby resulting in the deformation of the softer threads ensuring further the locking nature of the screw 300 relative to the plate 100. For example, thread 348 of screw 300 may be made of titanium alloy while threads 156/162 are made of a softer material such as commercially pure titanium.

Referring still to FIG. 3A, transfixation screw 300 may have a first recessed region proximate to the tip to form first cutting flute 170, and a second recessed region proximate to the head to form a second cutting flute 174. Cutting flutes 170 and 174 cut through a bone enabling screw 300 to bite into the bone to begin the engagement.

Referring to FIG. 4, a fixation plate 100 and transfixation screw 300 are shown following implant to fixate or attach first and second bones 404 and 408, respectively, across a joint 412. As shown, first and second bones 404 and 408, respectively, are of a human finger across a PIP joint 412. While FIG. 4 illustrates embodiments of the invention for attaching first and second bones across a PIP joint, it will be apparent to those skilled in the art that the invention may be used to attach bones across other joints within a human.

Referring still to FIG. 4, fixation plate 100 is placed upon first bone 404 following sectioning of bone 404 and is placed on one side of joint 412. It should be understood by those skilled in the art that embodiments of the invention may be configured to enable fixation plate 100 to span across joint 412.

Following drilling or placement of a K-wire or other well known guidance techniques, transfixation screw 300 is then inserted through screw hole 132 and rotated through the first bone 404, across the PIP joint and into the second bone 408. Nearing completion of the installation of screw 300, thread 328 engages bone 408 while thread 332 engages bone 404. Thus, it will be appreciated by those skilled in the art that due to the differing pitch of first and second threads, 328 and 332, respectively, transfixation screw 300 functions as a compression-type screw, firmly attaching or fixating first and second bones across joint 412. As the surgeon tightens screw 300 within the last bit of rotation within hole 132, locking threads 348 engage threads 156/162, deforming one relative to the other and locking screw 300 in place relative to plate 100 transfixing the first bone 404 relative to the second bone 408 across the PIP joint 412.

As discussed before, the trajectory of transfixation screw 300 is preliminarily determined by the angular orientation of central axis 152 relative to longitudinal axis 108 of between 20 and 50 degrees during the manufacture of plate 100. In addition, as noted above the present invention provides the surgeon with the flexibility of adjusting the orientation of screw 300 about the entire central axis 152 at an angle of between 0 and 18 degrees between the axis of the screw 300 and the central axis 152. Thus, within a plane parallel of the views shown in FIGS. 1B and 4, the orientation of transfixation screw 300 may be varied up to 18 degrees beyond the angular orientation of the central axis relative to the longitudinal axis 108. Consequently, the present invention allows the second bone 408 to be selectively positioned at a wide range of angles relative to first bone 404.

Referring still to FIG. 4, fixation plate 100 is secured to first bone 404 by a plurality of attachment screws 182, 186 and 190. As discussed before, depending on the length of the finger, the length of fixation plate 100 may be shortened by sawing or snipping off a portion thereof, which will reduce the number of attachment screws.

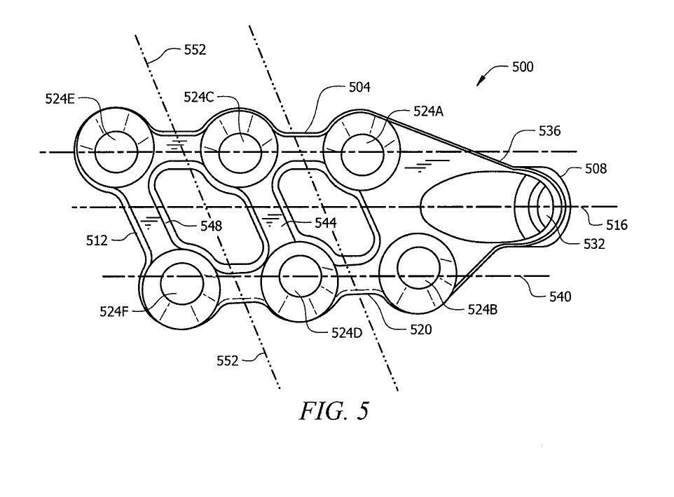

Referring now to FIG. 5, an alternate embodiment of a fixation plate 500 is shown having a body 504, a head 508, and a distal end 512, about a longitudinal axis 516. FIG. 5 illustrates top side 520 of fixation plate 500 which is intended to face away from a bone while bottom side is intended to be placed upon a bone. In some embodiments, fixation plate 500 is configured to be placed upon a bone and positioned entirely on one side of a joint.

Referring still to FIG. 5, fixation plate 500 includes locking screw hole 532 similar to that discussed above with respect to locking screw hole 132 in FIG. 2. However, in other embodiments of the invention screw hole 532 may not be locking.

Fixation plate 500 includes a plurality of attachment screw holes 524A-524F arranged in two rows along axes 536 and 540. Attachment screw holes 524C and 524D are interconnected via bridge 544, and attachment screw holes 524E and 524F are interconnected via bridge 548. By arranging the first and second group of attachment screw holes in two rows and interconnecting pairs of attachment screw holes via respective bridges, a grid configuration is formed. Depending on the size of a bone upon which fixation plate 500 is intended to be placed, the length of fixation plate 500 may be shortened by, for example, sawing or snipping off along line 552. Consequently, the number of attachment screw holes may be reduced depending on the size of a bone upon which fixation plate 500 is placed. Attachment screw holes 524A-524E may be locking or non-locking type. Plate 500 may include more attachment screw holes and more rows, thereby increasing the size of the grid of the plate 500 based on the teachings of the present invention.

Locking screw hole 532 may have a locking mechanism comprising a pair of threads arranged in a double helix configuration as discussed above with respect to locking screw hole 132 as shown in FIG. 2 or may feature any other suitable locking mechanism. Locking screw hole 532 is configured to lockably engage a transfixation screw such as screw 300 shown in FIGS. 3A and 3B. Thus, it will be appreciated that attachment screw holes 524A-524E receive respective attachment screws intended to firmly anchor fixation plate 500 to a first bone on one side of a joint while locking screw hole 532 is configured to receive a transfixation screw similar to screw 300 illustrated in FIGS. 3A and 3B which extends through the first bone, through a joint, and into a second bone, thereby fixating the second bone to the first bone.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A fixation plate, comprising:
a body having a longitudinal axis and first and second ends, the body configured to be placed upon a bone and positioned entirely on one side of a joint, the body having a locking screw hole disposed proximate to the first end, a central axis through the locking screw hole being oriented at an angle between 20 to 50 degrees relative to the longitudinal axis, the body having at least one attachment screw hole.

2. The fixation plate of claim 1, further comprising a locking attachment screw configured to be inserted through the attachment screw hole to lockably engage the attachment screw hole.

3. The fixation plate of claim 1, wherein an inner surface of the locking screw hole comprises an upper countersink and a threaded portion, the threaded portion having double helical threads.

4. The fixation plate of claim 3, wherein the threaded portion is configured to lockably engage a screw.

5. The fixation plate of claim 3, wherein the threaded portion is configured to deform when lockably engaging a screw.

6. The fixation plate of claim 1, wherein the body comprises upper and lower sides, and wherein the a portion of the lower side being contoured to be placed within a sectioned portion of a bone.

7. A fixation plate for securing first and second bones together across a joint, comprising:
a body having a longitudinal axis and first and second ends, the body configured to be placed upon the first bone and positioned on one side of the joint, a portion of the body proximate to the first end contoured to match the contour of the first bone when placed thereupon, the body having a locking screw hole disposed proximate to the first end, an inner surface of the locking screw hole having an upper countersink, a lower countersink and a threaded portion between the upper and lower countersinks, the threaded portion having double helical threads, a central axis through the locking screw hole being oriented at an angle between 20 to 50 degrees relative to the longitudinal axis, the body having at least one attachment screw hole.

8. The fixation plate of claim 7, wherein the second bone is positioned at an angle between 20 to 50 degrees relative to the longitudinal axis.

9. A screw, comprising:
an elongate hollow body having openings at first and second ends and having a tip at the first end and a head at the second end;
a first thread originating from the tip and extending proximate to the tip, the first thread having a first pitch;
a second thread disposed proximate to the head and having a second pitch, the second pitch being different than the first pitch; and
a locking thread disposed on the head and having a third pitch.

10. The screw of claim 9, wherein the elongate hollow body includes a smooth shank portion between the first thread and the second thread.

11. The screw of claim 9, wherein the locking thread and the second thread are separated.

12. The screw of claim 9, wherein the locking thread and the second thread are connected.

13. The screw of claim 9, further comprising a recessed region proximate to the tip to form a cutting flute.

14. The screw of claim 9, further comprising a recessed region proximate to the head to form a cutting flute.

15. A screw for securing first and second bones together across a joint, comprising:
an elongate hollow body having openings at first and second ends and having a tip at the first end and a head at the second end;
a first thread originating proximate the tip and extending proximally from the tip, the first thread having a first pitch;
a second thread disposed proximate the head, extending distally from the head and having a second pitch, the second pitch being different than the first pitch; and
a locking thread disposed on the head and having a third pitch,
wherein the screw is configured to extend from the first bone across the joint into the second bone to secure the second bone at an angle between 20 and 50 degrees relative to the first bone.

16. The screw of claim 15, wherein the elongate hollow body includes a smooth shank portion between the first thread and the second thread.

17. The screw of claim 15, wherein the locking thread and the second thread are separated.

18. The screw of claim 15, wherein the locking thread and the second thread are connected.

19. The screw of claim 15, further comprising a recessed region proximate to the tip to form a cutting flute.

20. The screw of claim 15, further comprising a recessed region proximate to the head to form a cutting flute.

21. A system for securing first and second bones together across a joint, comprising:
a fixation plate having a body, said body having a longitudinal axis and first and second ends, the body configured to be placed upon a bone and reside on one side of a joint, the body having a locking screw hole disposed proximate to the first end, a central axis through the locking screw hole oriented at a first angle between 20 to 50 degrees relative to the longitudinal axis, the body having at least one attachment screw hole; and
a screw having an elongate hollow body, said body having openings at first and second ends and having a tip at the first end and a head at the second end, the body configured to extend through the first bone across the joint and into the second bone, the head configured to abut the locking screw hole,
said screw further including:
a first thread originating proximate the tip and extending proximally to the tip, the first thread having a first pitch;
a second thread disposed proximate to the head, extending distally from the head and having a second pitch, the second pitch being different than the first pitch; and
a locking thread disposed on the head and having a third pitch, the locking thread configured to engage the locking screw hole.

22. The system of claim 21, wherein the elongate hollow body includes a smooth shank portion between the first thread and the second thread.

23. The system of claim 21, wherein the fixation plate being contoured proximate to the first end to match the contour of a sectioned portion of the first bone.

24. The system of claim 21, wherein said screw includes a recessed region proximate to the tip to form a cutting flute.

25. The system of claim 21, wherein said screw includes a recessed region proximate to the head to form a cutting flute.

26. The system of claim 21, wherein the central axis defines the trajectory of the screw.

27. The system of claim 21, wherein the second bone is positioned at an angle between 20 and 50 degrees relative to the longitudinal axis.

28. The system of claim 21, wherein the attachment screw hole is threaded to lockably engage a locking attachment screw.

29. The system of claim 21, wherein an inner surface of the locking screw hole has an upper counter sink and a threaded portion, the threaded portion having a pair of threads arranged in a double helical configuration.

30. The system of claim 21, wherein the first thread has a larger pitch than the second thread.

31. The system of claim 21, wherein the locking thread and the second thread are separated.

32. The system of claim 21, wherein the locking thread and the second thread are connected.

33. The system of claim 29, wherein the screw hole permits the threadable engagement with the screw at an offset angle from about 0 degrees to about 18 degrees off from the first angle.

34. A system for securing first and second bones together across a joint, comprising:
a fixation plate configured to be placed upon the first bone and reside on one side of the joint, the fixation plate comprising:
a body having a longitudinal axis and first and second ends, the body having a locking screw hole disposed proximate to the first end, an inner surface of the locking screw hole having an upper countersink, a lower countersink and a threaded portion between the upper and lower countersinks, the threaded portion having double helical threads, a central axis through the locking screw hole being oriented at an angle between 20 to 50 degrees relative to the longitudinal axis, the body having at least one attachment screw hole; and
a screw comprising:
an elongate hollow body having openings at first and second ends and having a tip at the first end and a head at the second end, the body configured to extend through the first bone across the joint and into the second bone, the head configured to abut the locking screw hole;
a first thread originating proximate the tip and extending proximally from the tip, the first thread having a first pitch;
a second thread disposed proximate to the head and having a second pitch, the second pitch being different than the first pitch; and
a locking thread disposed on the head and having a third pitch, the locking thread configured to engage the double helical threads on the locking screw hole when the screw is inserted through the locking screw hole,
wherein the second bone is positioned at an angle between 20 and 50 degrees relative to the longitudinal axis.

35. The system of claim 34, wherein the first thread has a larger pitch than the second thread.

36. The system of claim 34, wherein the locking thread and the second thread are separated.

37. The system of claim 34, wherein the locking thread and the second thread are connected.

38. The system of claim 34, wherein the double helical threads are configured to deform when lockably engaging the screw.

39. The system of claim 34, further comprising a recessed region proximate to the tip of the screw to form a cutting flute.

40. The system of claim 34, further comprising a recessed region proximate to the head of the screw to form a cutting flute.

41. A fixation plate, comprising:
a body connected to a head, the body having an axis and a rear end, the head having a locking screw hole oriented at an angle between 20 to 50 degrees relative to the axis, the body having a plurality of attachment screw holes arranged in at least two rows.

42. The fixation plate of claim 41, wherein a first row of attachment screw holes are arranged on a first side of the axis, and a second row of attachment screw holes are arranged on a second side of the axis.

43. The fixation plate of claim 41, wherein the attachment screw holes are configured to lockably engage respective locking attachment screws.

44. The fixation plate of claim 41, further comprising at least one bridge connecting a pair of the attachment screw holes across the axis.

45. The fixation plate of claim 41, wherein an inner surface of the locking screw hole comprises an upper countersink and a threaded portion, the threaded portion having double helical threads.

46. The fixation plate of claim 41, wherein the threaded portion is configured to lockably engage a screw.

47. The fixation plate of claim 41, wherein the threaded portion is configured to deform when lockably engaging a screw.

48. The fixation plate of claim 41, wherein the plate is configured to be placed on a bone and reside on one side of a joint.

49. A fixation plate, comprising:
a body connected to a head, the body having an axis and a rear end, the head having a locking screw hole oriented at an angle between 20 to 50 degrees relative to the axis, the body having a plurality of attachment screw holes arranged in two rows;
at least one bridge connecting a pair of the attachment screw holes across the axis,
wherein the fixation plate is configured to be placed on a bone and reside on one side of a joint.

50. The fixation plate of claim 49, wherein a first row of attachment screw holes are arranged on a first side of the axis, and a second row of attachment screw holes are arranged on a second side of the axis.

51. The fixation plate of claim 49, wherein the attachment screw holes are configured to lockably engage respective locking attachment screws.

52. The fixation plate of claim 49, wherein an inner surface of the locking screw hole comprises an upper countersink and a threaded portion, the threaded portion having double helical threads.

53. The fixation plate of claim 49, wherein the threaded portion is configured to lockably engage a screw.
